(19) United States
(12) Patent Application Publication
(10) Pub. No.: US 2011/0009746 A1
(43) Pub. Date: Jan. 13, 2011

(54) SYSTEM AND METHODS FOR PULMONARY EDEMA DETECTION WITH IMPLANTABLE ACOUSTIC DEVICES

(76) Inventors: Binh C. Tran, Minneapolis, MN (US); Bin Mi, Plymouth, MN (US); Yunlong Zhang, Mounds View, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/832,554

(22) Filed: Jul. 8, 2010

Related U.S. Application Data
(60) Provisional application No. 61/224,763, filed on Jul. 10, 2009.

(51) Int. Cl. A61B 8/00

(52) U.S. Cl. 600/437

(57) ABSTRACT

A system includes a first implantable acoustic transducer, a second implantable transducer a memory circuit, and a processor. The first implantable acoustic transducer is configured to receive transmitted acoustic energy from a thorax region of a subject and the second implantable acoustic transducer is configured to transmit the acoustic energy to the thorax region. The processor is communicatively coupled to the first acoustic transducer, the second acoustic transducer, and the memory circuit. The processor includes a parameter module configured to measure a parameter of the received acoustic energy, and a trending module configured to trend the measured parameter and to provide an indication of pulmonary edema status of the subject using the parameter trend.

### CROSS-REFERENCE TO RELATED APPLICATIONS

This application claims the benefit of U.S. Provisional Application No. 61/224,763, filed on Jul. 10, 2009, under 35 U.S.C. §119(e), which is hereby incorporated by reference in its entirety.

### TECHNICAL FIELD

This patent application pertains generally to implantable cardiac rhythm management devices and more particularly, but not by way of limitation, to systems and methods for monitoring pulmonary edema.

### BACKGROUND

Excess fluid retention in a subject can take various forms and can have different causes. Clinically, this fluid retention is called edema and can be classified as systemic or pulmonary edema. Examples of systemic edema include excess fluid accumulation in a subject's lower limbs, sacral area, abdominal cavity, or other parts of the body that receive blood through the aorta. Pulmonary edema involves a build-up of extravascular fluid in or around a subject's lungs.

One cause of pulmonary edema is congestive heart failure (CHF), sometimes referred to simply as “heart failure.” CHF can be conceptualized as an enlarged and weakened heart which results in lower cardiac stroke volume. As CHF worsens, increases in pulmonary circulatory pressure result in a build-up of fluid in body tissue. If the left side of the heart is impaired, fluid can accumulate in the lungs causing reduced capacity for ventilation and stiffening of the lungs, which may lead to respiratory failure. Monitoring fluid build up in the lungs can provide information concerning the progression of CHF.

### OVERVIEW

This document relates to systems and methods for monitoring hemodynamic function of a patient or subject, and in particular for improved monitoring of pulmonary edema.

A system example includes a first implantable acoustic transducer, a second implantable transducer, a memory circuit, and a processor. The first implantable acoustic transducer is configured to receive transmitted acoustic energy from a thorax region of a subject and the second implantable acoustic transducer is configured to transmit the acoustic energy to the thorax region. The processor is communicatively coupled to the first acoustic transducer, the second acoustic transducer, and the memory circuit. The processor includes a parameter module configured to measure a parameter of the received acoustic energy, and a trending module configured to trend the measured parameter and to provide an indication of pulmonary edema status of the subject using the parameter trend.

A method example includes receiving acoustic energy from a thorax region of a subject using a first implantable acoustic transducer, transmitting the acoustic energy to the thorax region using a second implantable acoustic transducer, trending a parameter of the received acoustic energy using a medical device, and indicating, to a user or process, a pulmonary edema status of the subject using the trended parameter.

This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of an example of portions of a system for monitoring excess fluid accumulation in the thoracic region of a subject.

FIG. 2 is an illustration of another example of portions of a system for monitoring excess fluid accumulation in the thoracic region of a subject.

FIG. 3 shows an illustration of still another example of portions of a system for monitoring excess fluid accumulation in the thorax region of a subject.

FIG. 4 shows an illustration of yet another example of portions of a system for monitoring excess fluid accumulation in the thorax region of a subject.

FIG. 5 shows a block diagram of portions of a method for monitoring excess fluid accumulation in the thorax region of a subject.

FIG. 6 shows a block diagram of portions of a system for monitoring excess fluid accumulation in the thorax region of a subject.

FIG. 7 shows a block diagram of portions of another system for monitoring excess fluid accumulation in the thorax region of a subject.

### DETAILED DESCRIPTION

This document discusses systems and methods for monitoring heart failure of a patient or subject. Specifically, systems to monitor fluid accumulation in the lungs are described.

As discussed above, CHF can result in a build-up of fluid in the thoracic region of a subject, such as in the lungs for example. As CHF worsens, cardiac output can continue to decrease resulting in more fluid build-up in the lungs. As the amount of fluid in the lung changes, acoustic properties of lung tissue change. Thus, changes in acoustic properties of the thoracic region can be correlated to changes in the amount of fluid accumulated in the lungs, and the acoustic properties can be used to track the progression of the subject's disease.

Changes in acoustic properties can be monitored by measuring changes in acoustic energy transmitted into the thorax region. Acoustic (e.g., ultrasonic) signals transmitted through a lung or lungs with accumulated fluid (i.e., a “wet lung”) may experience a modification that can be measured and tracked when the acoustic signal is received. For example, as the fluid level increases, an acoustic signal travelling through a wet lung region experiences less attenuation in amplitude than if an identical acoustic signal is passed through a normal lung. Therefore, by transmitting an acoustic signal of a known amplitude through the thoracic region, measuring the received amplitude, and analyzing the change in amplitude over time, a change in status of the patient's pulmonary edema can be detected.

Similarly, acoustic signals reflected from the region will experience less of a loss than if the fluid level was normal. Thus, pulmonary edema can also be monitored by transmitting an acoustic signal of a known amplitude into the thoracic region and monitoring the change in amplitude of the signal reflected from the region.

Another property that changes as fluid volume changes is the velocity of the acoustic signals. Acoustic signals travel faster through the thoracic region as the volume of fluid in the lungs increases. Thus, pulmonary edema can be further monitored by transmitting an acoustic signal at a known time and monitoring the change in transmission time between transmitting the signal and receiving the signal. Additionally, the increase in fluid volume can also shorten the propagation path of the signal. This can be viewed as the increased fluid forming a path of lesser resistance through the thoracic region for the acoustic signal to follow. This is another reason why pulmonary edema can be monitored by monitoring the change in transmission time.

A further property that changes as fluid volume changes is the attenuation of acoustic signals. The frequency of the transmitted acoustic signals can be swept through a range and the response measured to estimate the attenuation of the acoustic signal by the lung. Pulmonary edema can be monitored by changes in attenuation.

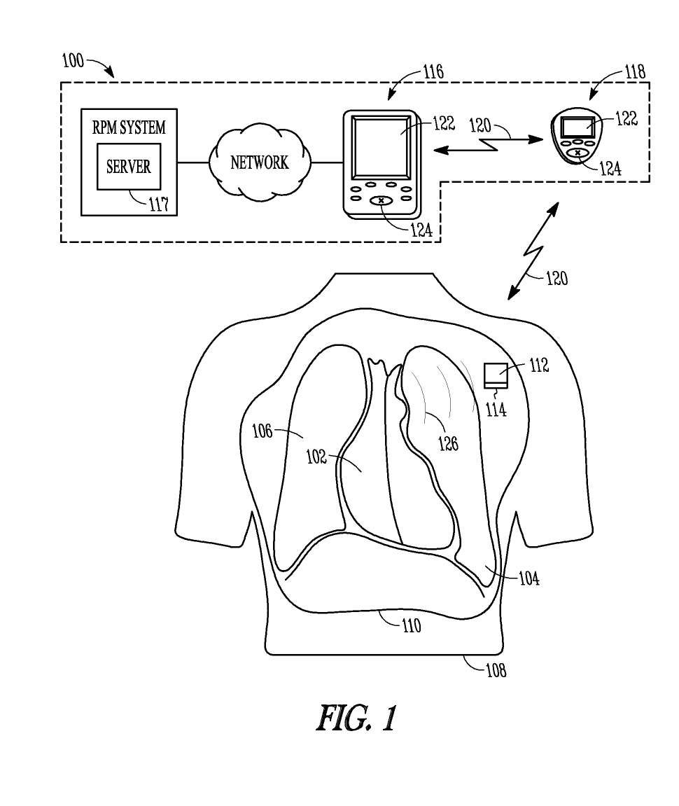

FIG. 1 is an illustration of an example of portions of a system 100 for monitoring excess fluid accumulation in the thoracic region of a subject 108. Also illustrated are the heart 102 and lungs 104 (left), 106 (right) of the subject 108 (via a cut-away portion 110). The system 100 includes a pectorally-implantable medical device (IMD) 112 including at least one acoustic transducer 114 and one or more programmers or other external user-interface devices 116, 118 providing wireless communication with the IMD 112, such as by using telemetry 120 or another communication technique.

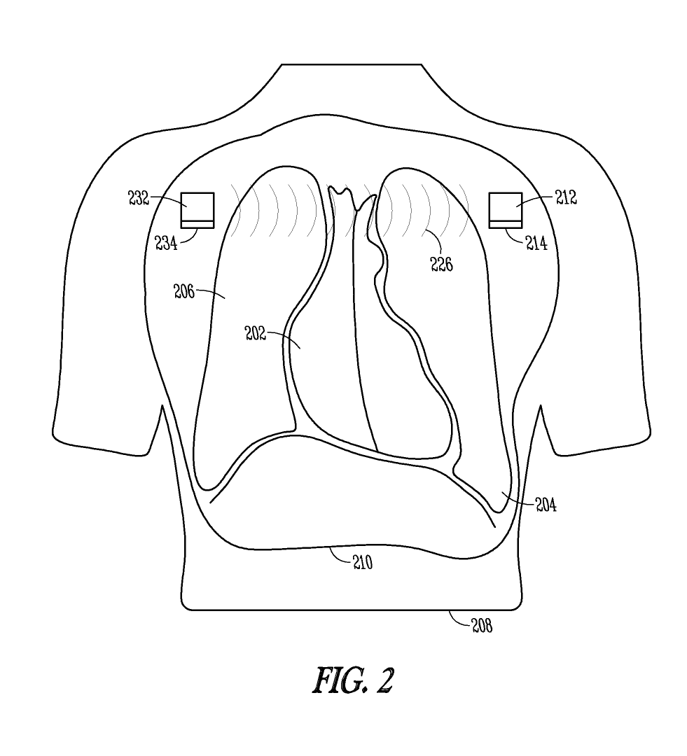

The acoustic transducer 114 is configured to receive transmitted acoustic energy 126. The acoustic energy 126 is transmitted through at least a portion of the thoracic region by a second device that may be external, or may also be implantable. For example, in FIG. 2, the IMD 212 is shown in the left pectoral region of the subject and a second IMD 232 is shown in the right pectoral region. Also shown are the heart 202 and lungs 204, 206 of the subject (via the cutaway portion 210). The second device 232 transmits acoustic energy 226 from an acoustic transducer 234 to the IMD 212 across the thorax region of the subject 208. Other device arrangements are contemplated. For example, the IMD 212 may be implanted in the left pectoral region while a second device 232 may be implanted in the heart or a vessel of the heart such as the pulmonary artery. Additional devices along with second device 232 may also be used to transmit acoustic energy 226 to IMD 212, allowing assessment over a larger lung volume. Similarly, additional devices along with IMD 212 may also be used to receive acoustic energy 226 transmitted by IMD 232. For example, there may be one or both of two transmitting devices and two receiving devices.

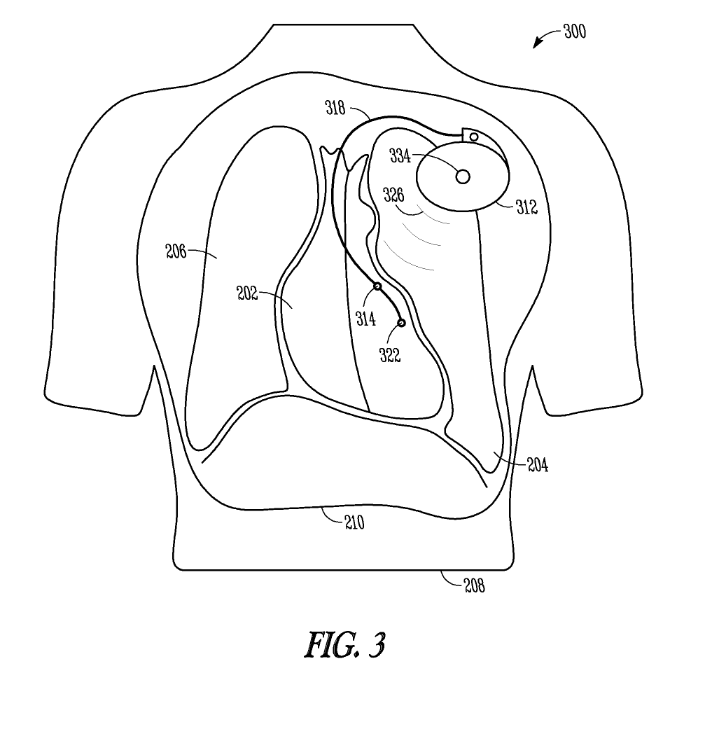

According to some examples, the acoustic energy may be transmitted and received using a single IMD. FIG. 3 shows an illustration of another example of a system 300 for monitoring excess fluid accumulation in the thorax region of a subject 208. The system 300 includes an IMD 312 that includes an acoustic transducer 334 integrated in the device housing. An implantable lead 318 is coupled to the IMD 312, such as by attachment to a header connector of the IMD 312. The implantable lead 318 may be a subcutaneous transducer bearing lead that includes at least a first transducer 314 to receive acoustic energy 326 transmitted by a second transducer 334. In the example shown, the lead placement allows for acoustic energy 326 to be transmitted across the left lung 304 portion of the thorax region of the subject 208. Other lead placements may be used. In some examples, multiple transducers may be arranged on the implantable lead 318 to receive the acoustic energy over greater lung area. In some examples, the IMD 312 is a cardiac function management (CFM) device and the implantable lead 318 includes at least one electrode 322 for one or both of sensing intrinsic cardiac activity and delivering electrical stimulation energy.

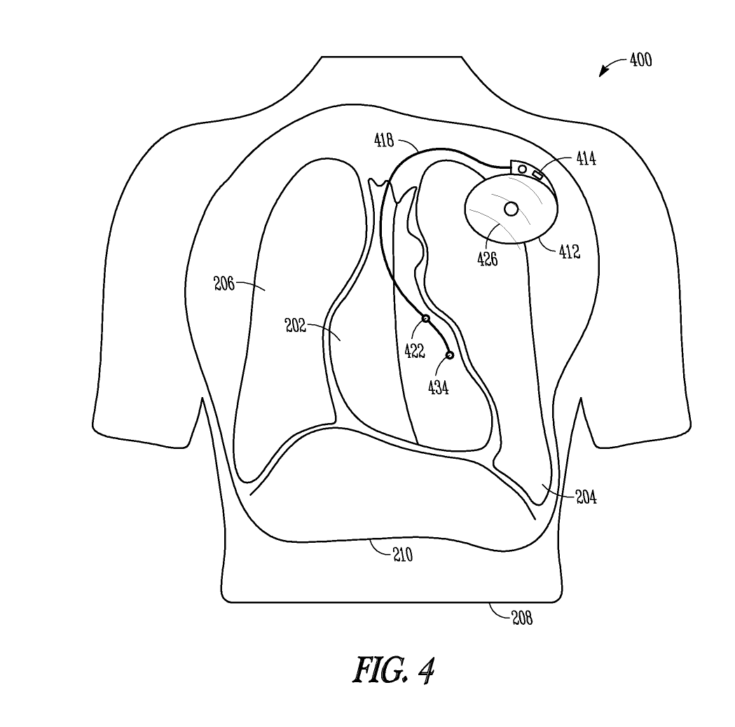

FIG. 4 shows an illustration of still another example of a system 400 for monitoring excess fluid accumulation in the thorax region of a subject 208. The system 400 again includes an IMD 412 coupled to an implantable lead 418 which may include at least one electrode 422. However, in this example, the implantable lead 418 includes an acoustic transducer 434 to transmit the acoustic energy 426 to the IMD 412, where another acoustic transducer 414 receives the transmitted acoustic energy 426. The acoustic transducer 414 may be integrated into the housing of the IMD 412, or as shown in the example, the acoustic transducer 414 may be integrated into a header connector of the implantable device 412. Again, an additional transducer or transducers on the implantable lead 418 allows the acoustic energy 326 to cover more lung area.

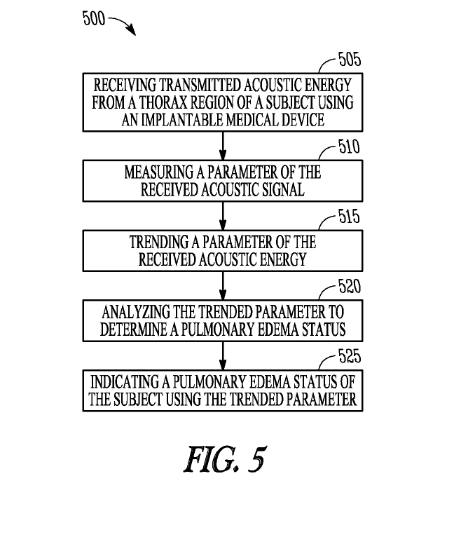

FIG. 5 shows a block diagram of portions of a method 500 for monitoring excess fluid accumulation in the thorax region of a subject. At block 505, transmitted acoustic energy is received from a thorax region of a subject using an IMD. At block 510, a parameter of the received acoustic energy is measured. A non-exhaustive list of examples of parameters that can be monitored by the IMD include a time interval from when the acoustic energy is transmitted to when the acoustic energy is received, an amplitude of the received acoustic energy, an attenuation of the received acoustic energy from the transmitted acoustic energy, and a frequency dependence of the received acoustic energy.

At block 515, the measured parameter of the received acoustic energy is trended over time. In the trending, measurements of the parameter are stored. Current measurements of the parameter are appended to previous measurements. The trending can be recurrent or periodic according to a programmable time period. Data is collected for an extended period, such as a week or a month or for a longer period.

At block 520, the trended parameter is analyzed to determine a pulmonary edema status. When a measurement of the parameter is taken, the collective data is used to determine the status, particularly to identify whether the status is trending towards worsening pulmonary edema. Whether the status is normal or abnormal, worsening or improving, is determined based on the history of the measurements. In certain examples, evaluation occurs on every measurement, and in certain examples, the evaluating is every nth measurement where n is an integer.

At block 525, an indication of a pulmonary edema status of the subject is determined using the trended parameter. In some examples, an indication of an abnormal status of pulmonary edema is provided to a user or process.

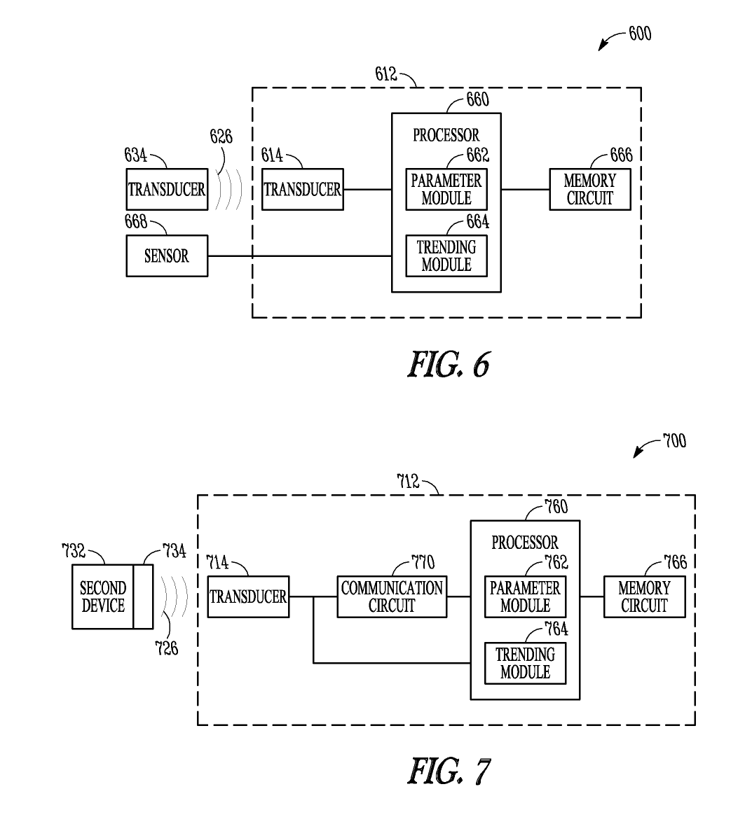

FIG. 6 shows a block diagram of portions of a system 600 for monitoring excess fluid accumulation in the thorax region of a subject. The system 600 includes a device 612 (e.g., an IMD) having a first implantable acoustic transducer 614 that receives transmitted acoustic energy 626 from a thorax region of a subject. In some examples, the acoustic energy is transmitted across the thorax region to the first implantable transducer using an external device.

In some examples, the acoustic energy is transmitted using a second implantable acoustic transducer 634. In certain examples, the second implantable acoustic transducer 634 is configured to transmit acoustic energy having a frequency in the range of 20 kilohertz (kHz) to 500 kHz and the first acoustic transducer is configured to receive acoustic energy having the frequency. In certain examples, the second implantable acoustic transducer 634 is configured to transmit acoustic energy having a frequency in the range of 1 kHz to 20 kHz and the first acoustic transducer is configured to receive acoustic energy having the frequency. In certain examples, the second implantable acoustic transducer 634 is configured to transmit acoustic energy having different amplitudes. Increasing the amplitude of the transmitted acoustic energy allows for deeper penetration and allows transmit paths to be longer.

The device 612 also includes a memory circuit 666 and a processor 660 that is communicatively coupled to the first acoustic transducer 614 and the memory circuit 666. The communicative coupling allows electrical signals to be communicated between the processor 660 and the first acoustic transducer 614, and between the processor 660 and the memory circuit 666, even though intervening circuitry may be present. The processor 660 may be a microprocessor, a digital signal processor, application specific integrated circuit (ASIC), or other type of processor. The processor 660 may include one or more modules to implement the functions described herein. Modules can be software, hardware, firmware or any combination thereof. The software and/or firmware are executed on the processor 660. Multiple functions can be performed in one or more modules as desired.

The processor 660 includes a parameter module 662 and a trending module 664. The parameter module 662 is configured to measure a parameter of the received acoustic energy, such as an amplitude of the received acoustic energy for example.

The trending module 664 is configured to trend the measured parameter. Measurements of the parameter are stored in the memory circuit 666. In some examples, the measurements are stored in a buffer where current measurements of the parameter are appended to previous parameter measurements. As described previously, parameter measurements are collected for an extended period of time (e.g., weeks or months). The trending includes evaluating the collected data for an indication of a trend (e.g. worsening or improving) of the subject's disease. The trending module 664 analyses the measurements of the parameter and provides an indication of pulmonary edema status of the subject using the parameter trend.

As shown in FIG. 1, the external user-interface devices 116, 118 can include, among other things, a user-detectable indication 122, such as an LCD or LED display, for textually or graphically relaying information about the pulmonary edema status derived from the trended parameter. The external user-interface devices 116, 118 can further include a user input device 124 configured for receiving programmable parameters from a user and communicating the parameters to the implantable device 112. In some examples, the external devices 116, 118 include a repeater to receive information from the IMD 112 and communicate the information over a network (e.g., a computer network or a telephone network) to another external device. This other external device may be a server 117 that is part of a remote patient management (RPM) system. The status of pulmonary edema may be communicated to a clinician via the RPM system.

Returning to FIG. 6, in certain examples, the acoustic energy is transmitted and received in pulses, and the parameter module 662 is configured to measure received transmissions of pulsed acoustic energy. In certain examples, the acoustic energy is transmitted and received continuously. Typically, a continuous transmission of acoustic energy is not transmitted indefinitely. The continuous transmission occurs over a time duration that is long compared to a respiration cycle of the subject, such as over a plurality of respiration cycles (e.g., three cycles) for instance. The parameter module 662 is configured to measure a received transmission of acoustic energy that is continuous over the plurality of respiration cycles.

According to some examples, the same device 612 (e.g., a single IMD) includes the first implantable acoustic transducer 614 and the second implantable acoustic transducer 634, which is also communicatively coupled to the processor 660. In some examples, the device 612 includes an implantable lead and the implantable lead includes the first implantable acoustic transducer 614, such as is shown in the example of FIG. 3 for instance. The first implantable acoustic transducer 614 receives acoustic energy at the implantable lead transmitted across the thorax region by the second implantable acoustic transducer 634 located within the body of the device 612. In other examples, the implantable lead includes the second acoustic transducer 634, such as is shown in FIG. 4 for instance. The first acoustic transducer 614 is located within the device 612 and receives acoustic energy 626 transmitted across the thorax region by the second acoustic transducer 634 located on the implantable lead.

The parameter module 662 measures a parameter of the received acoustic energy. As discussed previously, acoustic signals travel faster through the thoracic region as the volume of fluid in the lungs increases. Also, increased fluid in the lung may shorten the propagation path of the signal. Because the transducers are part of the same device, the processor can initiate the transmission and determine when the transmission is received. In some examples, the parameter module 662 measures a difference in the time interval from when the acoustic energy is transmitted by the second implantable acoustic transducer 634 to when the acoustic energy is received by the first implantable acoustic transducer 614. In certain examples, the parameter module 662 determines the velocity of the acoustic energy using the time interval and the distance between the first and second acoustic transducers. This distance may be programmed into the processor. The trending module 664 is configured to trend one or more of the time interval or the velocity and detect the occurrence of a significant change in the parameter. The trending module 664 provides an indication of pulmonary edema status using a trend of the measured interval or velocity. For example, the trending module 664 may provide an indication of worsening pulmonary edema if the timing interval decreases below an interval threshold or the velocity increases above a velocity threshold value.

An acoustic signal travelling through a wet lung region experiences less attenuation in amplitude than if an identical acoustic signal is passed through a normal lung. In some examples, the parameter module 662 measures an amplitude of the received acoustic energy. In certain examples, the parameter module 662 measures a central tendency (e.g., an average) of the amplitude. The trending module 664 is configured to trend a change in the amplitude of the received acoustic energy. The trending module 664 provides an indication of pulmonary edema status using a trend of the measured amplitude. For instance, the trending module 664 may provide an indication of worsening pulmonary edema when a change (e.g., a decrease) in the amplitude of the received acoustic energy signal differs from the transmitted amplitude by less than a threshold change value.

In certain examples, the parameter module 662 measures an attenuation of the received acoustic energy. The attenuation can be measured as one or more of a difference between the transmitted amplitude and the received amplitude and a ratio of the amplitudes. The trending module 664 is configured to trend a change in the attenuation of the received acoustic energy and provide an indication of pulmonary edema status using a trend of the measured attenuation. For instance, the trending module 664 may provide an indication of worsening pulmonary edema when the attenuation is less than a threshold attenuation value.

The effect of the lungs on acoustic signals of differing frequencies changes as fluid volume in the lungs changes. In some examples, the parameter module 662 measures a frequency dependence of the received acoustic energy. For instance, the parameter module 662 may be configured to sweep the frequency of the transmitted acoustic energy over a specified frequency range, such as 20 kHz-500 kHz or 1 kHz-20 kHz. In certain examples, the parameter module 662 measures the amplitude of the received acoustic energy at different frequencies to determine the relative change in amplitude at each frequency. In certain examples, the parameter module 662 detects a change in attenuation by evaluating the frequency dependence and detecting a change in slope of the amplitude versus frequency curve. The trending module 664 can be configured to trend the frequency dependence of the received acoustic energy or to trend the attenuation to provide an indication of pulmonary edema status. For instance, the trending module 664 may provide an indication of worsening pulmonary edema when the frequency dependence of a parameter of the acoustic energy signal changes by more than a specified threshold.

According to some examples, two separate devices (e.g., two IMDs) include the first implantable acoustic transducer 614 and the second implantable acoustic transducer 634, such as in the example shown in FIG. 2. Device 612 includes the first implantable acoustic transducer 614, the memory circuit 666, and the processor 660, and a second separate device includes the second implantable acoustic transducer 634. The parameter module 662 of the processor 660 in the first device 612 is configured to measure at least one of an amplitude of the received acoustic energy, and a frequency dependence of the received acoustic energy, by any of the methods described herein. The trending module 664 is configured to trend a change in one or more of the amplitude of the received acoustic energy, and the frequency dependence of the received acoustic energy. The trending module 664 determines a status of pulmonary edema using the trend and provides an indication of the status to a user or process.

According to some examples, the system 600 includes at least one sensor 668 communicatively coupled to the processor 660 and configured to provide an electrical sensor signal representative of a physiologic parameter of the subject. The sensor may be local to the device 612 or remote from the device 612. The trending module 664 is configured to determine the pulmonary edema status using the trended parameter and using the received sensor signal.

In some examples, the sensor 668 includes an implantable pressure sensor. In certain examples, the pressure sensor is implanted in a pulmonary artery. For instance, the pressure sensor may be affixed to a portion of the interior wall of the pulmonary artery to sense a signal representative of arterial pressure. In certain examples, the sensed pressure signal may be transmitted to the device 612 using a telemetry link. Examples of an implantable pressure sensor are described in U.S. Patent Publication No. US-2007-0088221, entitled, “Method and Apparatus for Pulmonary Artery Pressure Signal Isolation,” filed on Oct. 13, 2005, which is incorporated herein by reference in its entirety. The trending module 664 is configured to determine the pulmonary edema status using the trended acoustical signal parameter in conjunction with the pressure measurement from the sensor 668.

In some examples, the sensor 668 includes an implantable respiration sensor and provides a sensor signal representative of respiration. An example of an implantable respiration sensor is a transthoracic impedance sensor to measure minute respiration volume. An approach to measuring transthoracic impedance is described in Hartley et al., U.S. Pat. No. 6,076,015 “Rate Adaptive Cardiac Rhythm Management Device Using Transthoracic Impedance,” filed Feb. 27, 1998, which is incorporated herein by reference in its entirety. In some examples, the sensor 668 is an intra-thoracic impedance sensor (ITTI). A change in the respiration cycles of the subject may indicate a change in the pulmonary edema status of the subject. For example, an increased resting ventilation or respiration rate may indicate that the subject is having trouble breathing due to an increase in fluid in the lungs. The trending module 664 is configured to determine the pulmonary edema status using the trended parameter and using the respiration signal.

In some examples, the sensor 668 includes an implantable cardiac signal sensing circuit. The implantable cardiac signal sensing circuit produces a sensed electrical cardiac signal representative of cardiac activity of a subject. In some examples, the device 612 includes circuitry to detect heart rate from the sensed cardiac signal. In certain examples, such circuitry includes a peak detector circuit to detect R-waves corresponding to ventricular depolarizations. A change in the heart rate of the subject may indicate a change in the pulmonary edema status of the subject. For example, an increased resting heart rate may indicate that the heart rate is increasing due to an increase of fluid in the lungs. In some examples, the sensor 668 includes both a respiration sensor and a cardiac signal sensing circuit. The device 612 monitors both the subject's respiration rate and the heart rate in addition to the acoustic signal parameter to monitor pulmonary edema.

In some examples, the parameter module 662 is configured to measure the parameter of acoustic energy in relation to a specified phase of a respiration cycle of the subject. Measurements of the acoustic energy parameters may be modulated by the respiration cycle of the subject. Aligning the timing of the measurement to a specific phase of respiration is useful to remove variation in the measurement due to the subject's breathing.

According to some examples, a single device uses a single transducer to transmit and receive the acoustic energy. In FIG. 6, the first implantable acoustic transducer 614 is used to both to transmit acoustic energy to a thorax region of a subject and to receive acoustic energy reflected from the thorax region. Thus, the second implantable transducer 634 is not needed.

The parameter module 662 is configured to measure a parameter of the received reflected acoustic energy. The parameters measured may include, among other things, a time interval from when the acoustic energy is transmitted to when the reflected acoustic energy is received, an amplitude of the received reflected acoustic energy, and an attenuation of the received reflected acoustic energy from the transmitted energy. The trending module 664 is configured to trend a change in one or more of the time interval, the amplitude of the received reflected acoustic energy, or the attenuation of the received reflected acoustic energy.

In some examples, the processor is configured to transmit the acoustic energy in relation to a specified phase of a respiration cycle of the subject, such as a respiration cycle determined from an implantable respiration sensor. The parameter module is configured to measure the parameter of the reflected acoustic energy in relation to the specified phase.

FIG. 7 shows a block diagram of portions of another system 700 for monitoring excess fluid accumulation in the thorax region of a subject. The system 700 includes a device 712 (e.g., an IMD) having a first implantable acoustic transducer 714 that receives transmitted acoustic energy 726 from a thorax region of a subject. The device 712 also includes a memory circuit 766 and a processor 760. The processor 760 includes a parameter module 762 and a trending module 764.

The device 712 also includes a communication circuit 770 communicatively coupled to the first implantable acoustic transducer 714 and the processor 760. The communication circuit 770 is configured to extract an information signal from the received acoustic energy. The received acoustic energy is transmitted from a second device 732 having a second transducer 734. The second device 732 telemeters information using acoustic energy.

In some examples, the second device is an implantable device such as a pressure sensor implantable in the pulmonary artery and capable of communicating pressure data to the first device 712 via an acoustic telemetry link. An approach for communicating with an implanted device using acoustic telemetry can be found in Penner et al., U.S. Pat. No. 7,024,248, entitled “Systems and Methods for Communicating with Implantable Devices,” filed Nov. 19, 2001, which is incorporated herein by reference in its entirety. In some examples, the second device is an external device attachable to the patient's skin for communication with the first device 712 via an acoustic telemetry link.

The parameter module 762 is configured to measure a parameter of the received acoustic energy, such as an amplitude of the received acoustic energy for instance. The trending module is configured to trend a change in the measured (e.g., the amplitude) of the received acoustic energy, and to determine a status of pulmonary edema using the trend. An indication of the status is then provided by the trending module 764.

Increased fluid volume in lungs can be a symptom of heart failure. The acoustic properties of lung tissue change with the change in fluid volume. The described examples monitor the acoustic properties of the lungs and correlate the acoustic properties with fluid volume in order to monitor the progression of pulmonary edema in a subject.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventors also contemplate examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system comprising:
a first implantable acoustic transducer, wherein the first implantable acoustic transducer is configured to receive transmitted acoustic energy from a thorax region of a subject;
a second implantable acoustic transducer, wherein the second implantable acoustic transducer is configured to transmit the acoustic energy to the thorax region;
a memory circuit; and
a processor communicatively coupled to the first acoustic transducer, the second acoustic transducer, and the memory circuit, wherein the processor includes:
a parameter module configured to measure a parameter of the received acoustic energy; and
a trending module configured to trend the measured parameter and to provide an indication of pulmonary edema status of the subject using the parameter trend.

2. The system of claim 1, wherein a single implantable medical device (IMD) includes the first and second acoustic transducers.

3. The system of claim 2, wherein the parameter module is configured to measure at least one of:
a time interval from when the acoustic energy is transmitted to when the acoustic energy is received;
an amplitude of the received acoustic energy;
an attenuation of the received acoustic energy from the transmitted acoustic energy; and
a frequency dependence of the received acoustic energy, and
wherein the trending module is configured to trend a change in one or more of the time interval, the amplitude of the received acoustic energy, the attenuation of the received acoustic energy, and the frequency dependence of the received acoustic energy.

4. The system of claim 2, wherein the IMD includes an implantable lead, wherein the implantable lead includes the first implantable transducer.

5. The system of claim 2, wherein the IMD includes an implantable lead, wherein the implantable lead includes the second implantable transducer.

6. The system of claim 1, including:
a first device having the first implantable acoustic transducer, the memory circuit, and the processor; and
a second device having the second implantable acoustic transducer, and
wherein the parameter module of the processor in the first device is configured to measure at least one of:
an amplitude of the received acoustic energy; and
a frequency dependence of the received acoustic energy, and
wherein the trending module is configured to trend a change in one or more of the amplitude of the received acoustic energy, and the frequency dependence of the received acoustic energy.

7. The system of claim 1, wherein the parameter module is configured to measure received transmissions of pulsed acoustic energy.

8. The system of claim 1, wherein the parameter module is configured to measure a received transmission of acoustic energy that is continuous over a plurality of respiration cycles.

9. The system of claim 1, including an implantable respiration sensor communicatively coupled to the processor and configured to provide a sensor signal representative of respiration, and
wherein the parameter module is configured to measure the parameter of acoustic energy in relation to a specified phase of a respiration cycle of the subject.

10. A system comprising:
an implantable acoustic transducer configured to transmit acoustic energy to a thorax region of a subject and to receive acoustic energy reflected from the thorax region;
a memory circuit; and
a processor communicatively coupled to the implantable acoustic transducer and the memory circuit, wherein the processor includes:
a parameter module configured to measure a parameter of the received reflected acoustic energy; and
a trending module configured to trend the measured parameter and to provide an indication of pulmonary edema status of the subject using the parameter trend, and
wherein the parameter module is configured to measure at least one of:
a time interval from when the acoustic energy is transmitted to when the reflected acoustic energy is received;
an amplitude of the received reflected acoustic energy; and
an attenuation of the received reflected acoustic energy from the transmitted energy, and
wherein the trending module is configured to trend a change in one or more of the time interval, the amplitude of the received reflected acoustic energy, or the attenuation of the received reflected acoustic energy.

11. The system of claim 10, including an implantable respiration sensor communicatively coupled to the processor and configured to provide a sensor signal representative of respiration, and
wherein the parameter module is configured to measure the parameter of acoustic energy in relation to a specified phase of a respiration cycle of the subject.

12. A method comprising:
receiving acoustic energy from a thorax region of a subject using a first implantable acoustic transducer;
transmitting the acoustic energy to the thorax region using a second implantable acoustic transducer;
trending a parameter of the received acoustic energy using a medical device; and
indicating, to a user or process, a pulmonary edema status of the subject using the trended parameter.

13. The method of claim 12, including measuring at least one of:
an amplitude of the received acoustic energy;
an attenuation of the received acoustic energy from the transmitted acoustic energy; and
a time interval from when the acoustic energy is transmit to when the acoustic energy is received, and
wherein trending the parameter includes trending a change in one or more of the amplitude of the received acoustic energy, the attenuation of the received acoustic energy, or the time interval.

14. The method of claim 12, including:
sweeping a frequency of the transmitted acoustic energy over a specified frequency range using the IMD; and
measuring a frequency dependence of the received acoustic energy, and
wherein trending the parameter includes trending the frequency dependence of the received acoustic energy.

15. The method of claim 12, wherein transmitting the acoustic energy includes transmitting acoustic energy having a frequency in a range of 20 kHz to 500 kHz.

16. The method of claim 12, wherein transmitting the acoustic energy includes transmitting acoustic energy having a frequency in a range of 1 kHz to 20 kHz.

17. The method of claim 12,
wherein receiving acoustic energy includes receiving information telemetered using acoustic energy at a first device,
wherein transmitting acoustic energy includes transmitting the telemetered information to the first device using a second device,
wherein the method includes measuring an amplitude of the received acoustic energy, and
wherein trending the parameter includes trending a change in the amplitude measurement.

18. The method of claim 12, including:
receiving a sensor signal representative of at least one of pulmonary arterial pressure (PAP) and transthoracic impedance, and
wherein indicating a pulmonary edema status includes determining the pulmonary edema status using the trended parameter and the sensor signal.

19. The method of claim 12, including:
determining a respiration cycle of the subject using a received sensor signal that is representative of respiration, and
wherein trending the parameter includes measuring the parameter in relation to a specified phase of a respiration cycle of the subject.

20. A method comprising:
transmitting acoustic energy to a thorax region of a subject using an implantable acoustic transducer;
receiving a reflection of the transmitted acoustic energy at the implantable acoustic transducer;
measuring, using a medical device, at least one of:
an amplitude of the received reflected acoustic energy;
an attenuation of the received reflected acoustic energy from the transmitted acoustic energy; and
a time interval from when the acoustic energy is transmit to when the reflected acoustic energy is received;
trending a change in one or more of the amplitude of the received reflected acoustic energy, the attenuation of the received reflected acoustic energy, and the time interval; and
indicating, to a user or process, a pulmonary edema status of the subject using the trend.
